(19) United States
(12) Patent Application Publication
(10) Pub. No.: US 2004/0057918 A1
(43) Pub. Date: Mar. 25, 2004

(54) Photostabilized dibenzoylmethane sunscreens immobilized within sol/gel matrices

(75) Inventor: Sandrine Chodorowski-Kimmes, Senlis (FR)

Correspondence Address: BURNS, DOANE, SWECKER & MATHIS, L.L.P., P.O. Box 1404, Alexandria, VA, 22313-1404, US

(73) Assignee: SOCIETE L'OREAL S.A., Paris (FR)

(21) Appl. No.: 10/621,507

(22) Filed: Jul. 18, 2003

Related U.S. Application Data
(60) Provisional application No. 60/397,616, filed on Jul. 23, 2002.

(30) Foreign Application Priority Data
Jul. 19, 2002 (FR) 02/09210

(51) Int. Cl. A61K 7/42

(52) U.S. Cl. 424/59

(57) ABSTRACT

Photoprotective cosmetic/dermatological compositions well suited for the UV-photoprotection of the skin, hair, nails, eyebrows, eyelashes and/or lips, contain an effective photostable and UV-photoprotecting amount of at least one dibenzoylmethane organic sunscreen immobilized within a matrix prepared via sol-gel process from at least one silicon alkoxide, at least one non-ionic surfactant and water, but in the absence of any organic solvent, formulated into a topically applicable, cosmetically/dermatologically acceptable medium therefor.

### CROSS-REFERENCE TO PRIORITY/PROVISIONAL APPLICATIONS

This application claims priority under 35 U.S.C. § 119 of FR-02/09210, filed Jul. 19, 2002, and of provisional application Serial No. 60/397,616, filed Jul. 23, 2002, both hereby expressly incorporated by reference. This application is also a continuation of said '616 provisional.

### BACKGROUND OF THE INVENTION

1. Technical Field of the Invention

The present invention relates to a process for photostabilizing an organic sunscreen agent of dibenzoylmethane type, and a sol-gel process for preparing a material comprising such a sunscreen agent. It also relates to the resulting material and a cosmetic and/or dermatological composition containing such a material.

2. Description of Background/Related/Prior Art

It is known that light radiation with wavelengths of between 280 nm to 400 nm permits tanning of the human epidermis, and that radiation of wavelengths of between 280 nm to 320 nm, known as UV-B radiation, causes erythemas and skin burns which can be harmful to the development of natural tanning; this UV-B radiation must therefore be screened out.

It is also known that UV-A radiation of wavelengths between 320 nm and 400 nm, which results in tanning of the skin, is capable of causing damage thereto, in particular in the case of sensitive skin or of skin continually exposed to solar radiation. UV-A radiation is more penetrating than UV-B radiation and causes, in particular, loss of elasticity of the skin and the appearance of wrinkles, leading to premature aging. UV-A radiation promotes the onset of the erythema reaction or enhances this reaction in certain individuals, and may even be responsible for phototoxic or photoallergic reactions. It is therefore desirable to also screen out UV-A radiation and in particular long UV-A.

Therefore, for the purpose of ensuring protection for the skin and keratinous materials against UV radiation, sunscreen compositions comprising filters which are active in the UV-A region and filters which are active in the UV-B region are generally used.

In this respect, a well-known and particularly significant class of UV-A active filters is constituted by the dibenzoylmethane derivatives, and in particular 4-(tert-butyl)-4′-methoxydibenzoylmethane commercially available under the trademark “PARSOL® 1789” by HOFFMANN LAROCHE.

Nonetheless, sunscreens such as Parsol® 1789 have a major problem of sensitivity to UV radiation. In fact, dibenzoylmethane derivatives are compounds which are relatively sensitive to ultraviolet radiation. They have an unfortunate tendency to decompose more or less rapidly under the action of the latter. Thus, this photochemical sensitivity of dibenzoylmethane derivatives under the influence of ultraviolet radiation, to which they are naturally intended to be subjected, does not permit a guarantee of constant protection during prolonged exposure to the sun, such that repeated applications at regular and frequent intervals of time are necessary in order to provide the user with effective protection of the skin against UV rays.

Consequently, attempts have been made to reduce the sensitivity to UV radiation of these filters, but numerous formulation problems have been encountered. For example, these particular photosensitive filters cannot be formulated with some other active products, requiring in each case the development of a new formulation. Furthermore, these photosensitive filters have a protective effect necessarily limited in time and, to compensate their sensitivity to UV radiation, it is necessary to include significant quantities thereof in formulations without this being enough to solve the problem.

This problem of photoinstability of sunscreen agents of dibenzoylmethane type is well known to this art.

Indeed, FR-A-2,799, 119 describes a process for stabilizing with respect to UV radiation a dibenzoylmethane type photosensitive sunscreen agent by incorporating it into the matrix prepared by the sol-gel route from at least one silicon alkoxide, at least one solvent, at least one surfactant and water. However, improvement in the photostability of these filters is not always satisfactory.

### SUMMARY OF THE INVENTION

The application has now surprisingly and unexpectedly been determined that immobilization of a dibenzoylmethane organic sunscreen agent in a matrix prepared by the sol-gel route from at least one silicon alkoxide, at least one surfactant and water, without addition of any organic solvent, results in a product having better photostability.

Other advantageous properties are also obtained with this novel solar material. In particular, the material and the compositions comprising it are able to form films, impart a pleasant touch and exhibit very good persistence toward pure or saline water and toward polar and non-polar cosmetic oils.

The present invention thus features a process of photostabilization of a dibenzoylmethane organic sunscreen agent.

The present invention also features a process for preparing a material containing a dibenzoylmethane organic filter via the sol-gel route.

This invention also features a material obtainable by the preparation process thereof.

Yet another feature of the present invention is a cosmetic and/or dermatological composition comprising such a material.

Other characteristics, aspects, features and advantages of the invention will become more clearly apparent on reading the description and various examples which follows.

### DETAILED DESCRIPTION OF BEST MODE AND SPECIFIC/PREFERRED EMBODIMENTS OF THE INVENTION

The process for photostabilizing a dibenzoylmethane type organic sunscreen agent according to the present invention entails immobilizing said sunscreen agent in a matrix prepared by the sol-gel route from at least one silicon alkoxide, at least one surfactant and water, without adding organic solvent.

Dibenzoylmethane organic sunscreen agent is understood to mean any compound in the form of a simple molecule, dimer, oligomer or polymer, having at least one dibenzoylmethane chromophoric group.

Non-limiting examples of dibenzoylmethane type organic sunscreen agents which can be used within the scope of the present invention, especially, are

2-methyldibenzoylmethane,

4-methyldibenzoylmethane,

4-isopropyldibenzoylmethane, or Isopropyl Dibenzoylmethane, such as that marketed as “EUSOLEX® 8020” by MERCK,

4-tert-butyldibenzoylmethane,

2,4-dimethyldibenzoylmethane,

2, 5-dimethyldibenzoylmethane,

4,4′-diisopropyldibenzoylmethane,

4,4′-dimethyldibenzoylmethane,

4-tert-butyl-4′-methoxydibenzoylmethane, or Butyl Methoxydibenzoylmethane,

2-methyl-5-isopropyl-4′-methoxydibenzoylmethane,

2-methyl-5-tert-butyl-4′-methoxydibenzoylmethane,

2,4-dimethyl-4′-methoxydibenzoylmethane,

2, 6-dimethyl-4-tert-butyl-4′-methoxydibenzoyl-methane.

Butyl Methoxydibenzoylmethane marketed especially under the brand name “PARSOL® 1789” by HOFFMANN-LA ROCHE, represented by the following developed formula, is preferably used as dibenzoylmethane type organic sunscreen agent,

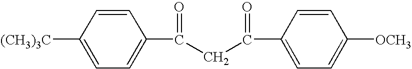

The silicon alkoxides used in the process according to the present invention are selected especially from those represented by the following formulae:

Si(ORₐ)₄ (I)

R—Si(ORₐ)₃ (II)

(RₐO)₃—Si—R″—Si(ORₐ)₃ (III)

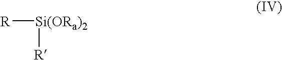

in which

Rₐ represents a linear or branched, C₁-C₃₀, preferably C₁-C₆, alkyl group;

R and R′, independently of one another, represent a linear or branched alkyl group, a cycloalkyl group, a substituted or unsubstituted aryl group, said groups R and R′ being able to further comprise a cosmetically or dermatologically active group;

R″ represents a linear or branched alkylene group, a cycloalkylene group, a substituted or unsubstituted arylene group, said group R″ being able to further comprise a cosmetically or dermatologically active group.

Among the cosmetically or dermatologically active groups which can be carried by the R, R′ and R″ groups, mention can be made, for example, of a coloring group, a photochromic group, a group which promotes adherence on keratinous materials such as groups of phosphate, phosphonate, phosphonic acid, amide, urethane, ureide, hydroxy, carboxy, thiol, amino acid or polypeptide type, a group for combating free radicals or a vitamin-carrying group.

Tetraethoxysilane and methyltriethoxysilane are particularly preferred among the silicon alkoxides defined hereinabove.

The surfactants used in the process according to the present invention are preferably non-ionic. They are especially selected from polyethoxylated, polypropoxylated or polyglycerolated fatty alcohols, alkylphenols or acids with a fatty chain comprising from 6 to 32 carbon atoms, preferably from 8 to 18 carbon atoms, the number of ethylene oxide or propylene oxide groups being between 2 and 100, preferably between 2 and 50 and the number of glycerol groups being between 2 and 30.

Other examples of non-ionic surfactants are ethylene and propylene oxide copolymers; condensates of ethylene and propylene oxides with fatty alcohols; polyethoxylated fatty amides preferably having 2 to 30 moles of ethylene oxide; polyglycerolated fatty amides comprising preferably 1 to 5 glycerol groups and in particular 1.5 to 4; polyethoxylated fatty amines preferably having 2 to 30 moles of ethylene oxide; sorbitan fatty acid esters ethoxylated with 2 to 30 moles of ethylene oxide; sucrose fatty acid esters, polyethylene glycol fatty acid esters, alkylpolyglucosides, carbamate or amide derivatives of N-alkylglucamines, aldobionamides, amine oxides such as alkylamine or N-acylamidopropylmorpholine oxides.

Among the above-mentioned surfactants, preference is given in particular to polyethoxylated 4-(1,1,3,3-tetramethylbutyl)phenol. The latter is marketed under the name Triton® X100 by Union Carbide.

The present invention also features a process for preparing a material containing a dibenzoylmethane organic sunscreen agent, by the sol-gel route, comprising mixing at least one dibenzoylmethane organic sunscreen agent, at least one surfactant, at least one silicon alkoxide and water in a quantity sufficient for partial or total hydrolysis of the alkoxide and its condensation, in the absence of organic solvent.

The organic sunscreen agent of dibenzoylmethane type, surfactants, and silicon alkoxides used in said preparation process are as described hereinabove.

The starting compounds, that is, the surfactants and the silicon alkoxides, as well as their quantities and the quantity of water, will be selected for a given organic sunscreen agent of dibenzoylmethane type such that said filter is dispersed homogeneously in the final material.

Said sunscreen agents are especially used in the process of the present invention in a quantity preferably ranging from 0.1% to 20% by weight, better still from 0.5% to 10% by weight relative to the weight of the silicon alkoxide.

The quantity of surfactant used in the scope of the invention depends on the solubility of the surfactant in an alkoxide/water mixture. It is such that the weight ratio of the surfactant to the silicon alkoxide is especially between 0.01 and 99, and preferably between 1 and 60.

Water is used in a quantity sufficient for total or partial hydrolysis of the silicon alkoxide and its condensation. This quantity is between 0.01% and 99% by weight, preferably between 1% and 50% by weight relative to the weight of the silicon alkoxide.

The water preferably comprises a cosmetically acceptable mineral or organic acid, and more particularly hydrochloric acid, nitric acid, sulfuric acid or acetic acid. Within the scope of the present invention hydrochloric acid is particularly preferred.

The pH of the hydrolysis water preferably ranges from 1 to 4, better still from 1 to 2.

A preferred embodiment of the preparation process as defined hereinabove comprises adding to the mixture of at least one surfactant, preferably non-ionic, and of a sunscreen agent of dibenzoylmethane type, silicon alkoxide in the first instance, and then water containing a cosmetically acceptable mineral or organic acid.

The invention also features the material containing a dibenzoylmethane organic sunscreen agent obtainable by the above-described preparation process.

The present invention also features a cosmetic and/or dermatological composition comprising, in a cosmetically and/or dermatologically acceptable medium, an effective quantity of at least one material containing a dibenzoylmethane type organic sunscreen agent obtainable by the preparation process of the present invention.

Cosmetically and/or dermatologically acceptable medium is understood to be a medium compatible with all the keratinous materials such as skin, hair, nails, eyelashes and eyebrows, lips and any other zone of the body and face.

This cosmetic and/or dermatological composition comprises said material in an effective quantity ranging from 1% by weight to 99% by weight, preferably from 1% by weight to 60% by weight, relative to the total weight of the composition.

Preferably, the inventive material is dried and ground, and added in the form of particles to a cosmetic and/or dermatological composition. The average particle size depends on the type of grinding method and the apparatus used and is advantageously in the range from 0.1 to 50 μm, preferably from 0.1 to 20 μm, and better still from 0.1 to 10 μm.

The cosmetic and/or dermatological compositions according to the present invention may also contain one or more additional organic sunscreen agents which are active in the UV-A and/or UV-B region, other than the dibenzoylmethane sunscreens defined hereinabove, these additional sunscreen agents being water-soluble, fat-soluble, or insoluble in the usual cosmetic solvents, provided of course that they do not alter the essential characteristic of these compositions, namely their photostability.

These additional filters can be especially selected from anthranilates; salicylic derivatives; cinnamic derivatives; camphor derivatives; triazine derivatives such as those described in U.S. Pat. No. 4,367,390, EP-0-863,145, EP-0-517,104, EP-0-570,838, EP-0-796,851, EP-0-775,698, EP-0-878,469 and EP-0-933,376; benzophenone derivatives; aminated 2-hydroxybenzophenones such as those described in EP-1-046,391 and DE-10-012,408; β,β′-diphenylacrylate derivatives; benzotriazole derivatives; benzalmalonate derivatives; benzimidazole derivatives; imidazolines; bis(benzoazolyl) derivatives such as those described in EP-0-669,323 and U.S. Pat. No. 2,463,264; p-aminobenzoic acid (PABA) derivatives; methylene-bis(hydroxyphenyl-benzotriazole) derivatives such as those described in U.S. Pat. Nos. 5,237,071, 5,166,355, GB-2,303,549, DE-19-726,184 and EP-0-893,119; hydrocarbon polymer filters and silicone filters described in WO-93/104665; alkylstyrene derivatives such as those described in DE-198-55,649; and their mixtures.

The following compounds designated by their INCI name, as well as their mixtures, can be cited by way of example of such additional organic sunscreen agents which are active in the UV-A and/or UV-B regions:

Anthranilates:

Menthyl Anthranilate sold under the brand name NEO HELIOPAN® MA by HAARMAN ET REIMER;

Salicylic Derivatives:

homosalate sold under the brand name EUSOLEX® HMS by RONA/EM INDUSTRIES,

Ethylhexyl Salicylate sold under the brand name NEO HELIOPAN® OS by HAARMANN ET REIMER,

Dipropyleneglycol Salicylate sold under the brand name DIPSAL® by SCHER,

TEA Salicylate sold under the brand name NEO HELIOPANS® TS by HAARMANN ET REIMER;

Cinnamic Derivatives:

Ethylhexyl Methoxycinnamate sold especially under the brand name PARSOL® MCX by HOFFMANN LA ROCHE,

Isopropyl Methoxycinnamate,

Isoamyl Methoxycinnamate sold under the brand name NEO HELIOPAN® 1000 by HAARMANN ET REIMER,

Cinnoxate,

DEA Methoxycinnamate,

Diisopropyl Methylcinnamate,

Glyceryl Ethylhexanoate Dimethoxycinnamate;

Camphor Derivatives:

3-Benzylidene Camphor manufactured under the name MEXORYL® SD by CHIMEX,

Benzylidene Camphor Sulfonic Acid manufactured under the name MEXORYL® SL by CHIMEX,

Camphor Benzalkonium Methosulfate manufactured under the name MEXORYL® SO by CHIMEX,

Terephthalylidene Dicamphor Sulfonic Acid manufactured under the name MEXORYL® SX by CHIMEX,

Polyacrylamidomethyl Benzylidene Camphor manufactured under the name MEXORYL® SW by CHIMEX;

Triazine Derivatives:

Anisotriazine sold under the brand name TINOSORB® S by CIBA GEIGY,

Ethylhexyl Triazone sold especially under the brand name UVINUL® T150 by BASF,

Diethylhexyl Butamido Triazone sold under the brand name UVASORB® HEB by SIGMA 3V,

2,4,6-tris-(diisobutyl 4′-aminobenzalmalonate)-s-triazine;

Benzophenone Derivatives:

Benzophenone-1 sold under the brand name UVINUL® 400 by BASF,

Benzophenone-2 sold under the brand name UVINUL® D-50 by BASF,

Benzophenone-3 or Oxybenzone sold under the brand name UVINUL® M-40 by BASF,

Benzophenone-4 sold under the brand name UVINUL® MS-40 by BASF,

Benzophenone-5,

Benzophenone-6 sold under the brand name HELISORB® 11 by NORQUAY,

Benzophenone-8 sold under the brand name SPECTRA-SORB® UV-24 by AMERICAN CYANAMID,

Benzophenone-9 sold under the brand name UVINUL® DS-49 by BASF,

Benzophenone-12,

n-hexyl 2-(4-diethylamino-2-hydroxybenzoyl)-benzoate;

β,β′-Diphenylacrylate Derivatives:

Octocrylene sold especially under the brand name UVINUL® N539 by BASF,

Ethocrylene, sold especially under the brand name UVINUL® N35 by BASF;

Benzotriazole Derivatives:

Drometrizole trisiloxane sold under the brand name SILATRIZOLE® by RHODIA CHIMIE,

Methylene bis-Benzotriazolyl Tetramethylbutyl-phenol sold in solid form under the brand name MIXXIM® BB/100 by FAIRMOUNT CHEMICAL or in micronised form in aqueous dispersion under the brand name TINOSORB® M by CIBA SPECIALTY CHEMICALS;

Benzalmalonate Derivatives:

polyorganosiloxane with benzalmalonate functions sold under the brand name PARSOL® SLX by HOFFMANN LA ROCHE;

Benzimidazole Derivatives:

Phenylbenzimidazole Sulfonic Acid sold especially under the brand name EUSOLEX® 232 by MERCK,

Disodium Phenyl Dibenzimidazole Tetra-sulfonate sold under the brand name NEO HELIOPAN® AP by HAARMANN and REIMER;

Inidazoline Derivatives:

Ethylhexyl Dimethoxybenzylidene Dioxoimidazoline Propionate;

Para-Aminobenzoic Acid Derivatives:

PABA,

Ethyl PABA,

Ethyl Dihydroxypropyl PABA,

Ethylhexyl Dimethyl PABA sold under the brand name ESCALOL® 507 by ISP,

Glyceryl PABA,

PEG-25 PABA sold under the brand name UVINUL® P25 by BASF.

Additional organic UV filters which are more particularly preferred are selected from the following compounds:

Ethylhexyl Salicylate,

Octocrylene,

Phenylbenzimidazole Sulfonic Acid,

Terephthalylidene Dicamphor Sulfonic Acid,

4-Methylbenzylidene Camphor,

Anisotriazine,

Ethylhexyl Triazone,

Diethylhexyl Butamido Triazone,

2,4,6-tris-(diisobutyl 4′-amino-benzalmalonate)-s-triazine,

Benzophenone-3,

Benzophenone-4,

Benzophenone-5,

n-hexyl 2-(4-diethylamino-2-hydroxybenzoyl)-benzoate,

Methylene bis-Benzotriazolyl Tetramethylbutyl-phenol,

Drometrizol Trisiloxane,

Disodium Phenyl Dibenzimidazole Tetra-sulfonate, and their mixtures.

The cosmetic and/or dermatological compositions according to the invention can also contain pigments or alternatively nanopigments (average primary particle size of generally between 5 nm and 100 nm, preferably between 10 and 50 mm) formed of coated or uncoated metal oxides such as for example, nanopigments of titanium oxide (amorphous or crystalline in the rutile and/or anatase form), iron oxide, zinc oxide, zirconium oxide or cerium oxide which are all photoprotective agents well-known per se. Conventional coating agents are alumina and/or aluminum stearate. Such nanopigments of coated or uncoated metal oxides are described in particular in EP-A-0-518,772 and EP-A-0-518,773.

The compositions according to the invention may also contain artificial browning and/or tanning agents for skin (self-tanning agents) such as, for example, dihydroxyacetone (DHA).

The compositions according to the present invention can further comprise conventional cosmetic additives, especially selected from fatty substances, organic solvents, ionic or non-ionic thickeners, softeners, antioxidants, agents for combating free radicals, opacifiers, stabilizers, emollients, silicones, α-hydroxy acids, anti-foaming agents, moisturizing agents, vitamins, insect repellents, perfumes, preservatives, anionic, cationic, amphoteric or non-ionic surfactants, anti-inflammatories, substance P antagonists, fillers, sequestering agents, polymers, propellants, basifying or acidifying agents, dyes or any other ingredient usually used in the cosmetic and/or dermatological field, in particular for manufacturing sunscreen compositions in the form of emulsions, and their mixtures.

The fatty substances can be constituted by an oil or a wax or their mixtures. “Oil” is understood as a compound which is liquid at ambient temperature. “Wax” is understood as a compound which is solid or substantially solid at ambient temperature, and which has a melting point generally of greater than 35° C. They also comprise linear or cyclic fatty acids, fatty alcohols and fatty acid esters, such as derivatives of benzoic, trimellitic and hydroxybenzoic acids.

Examples of oils are mineral oils (Vaseline); vegetable oils (sweet almond oil, macadamia oil, blackcurrant seed oil, jojoba oil); synthetic oils such as perhydrosqualene, fatty alcohols, fatty acids or fatty esters (such as C₁₂-C₁₅ alcohol benzoate sold under the brand name “Finsolv TN” by Finetex, octyl palmitate, isopropyl lanolate, triglycerides including those of capric/caprylic acids), ethoxylated or propoxylated fatty esters and ethers; silicone oils (cyclomethicone preferably having 4 or 5 silicon atoms, polydimethylsiloxane or PDMS) or fluorinated oils, or polyalkylene oils.

Examples of waxy compounds are paraffin, carnauba wax, beeswax, hydrogenated castor oil.

Examples of organic solvents are lower alcohols and polyols.

Thickeners can be selected in particular from crosslinked polyacrylic acids, modified or unmodified guar and cellulose gums, such as hydroxypropylated guar gum, methylhydroxyethylcellulose, hydroxypropylmethylcellulose, and silicone gums, such as for example, a polydimethylsiloxane derivative.

The cosmetic and/or dermatological compositions according to the invention can be prepared according to techniques well known to this art, in particular those intended for preparation of emulsions of oil-in-water or water-in-oil type.

These cosmetic and/or dermatological compositions may in particular be provided in the form of simple or complex emulsion (O/W, W/O, O/W/0 or W/O/W) such as a cream, a milk, or in the form of a gel or a gel cream, powder, solid stick and can optionally be packaged in an aerosol and be provided in the form of foam or spray.

When this is an emulsion, the aqueous phase thereof can comprise a non-ionic vesicular dispersion prepared according to known processes (Bangham, Standish and Watkins, J. Mol. Biol. 13, 238 (1965), FR 2,315,991 and FR 2,416,008).

The cosmetic and/or dermatological composition according to the invention can be used as composition for protecting keratinous materials such as skin, hair, nails, lips, eyelashes or eyebrows, against ultraviolet rays, as a sunscreen composition or as a makeup product.

When the cosmetic composition according to the invention is used to protect the skin against UV rays or as a sunscreen composition, it can be provided in the form of a suspension or dispersion in solvents or fatty substances, in the form of a non-ionic vesicular dispersion, or alternatively of an emulsion, preferably, of the oil-in-water type, such as a cream or a milk, or in the form of pomade, gel, gel cream, solid stick, powder, stick, aerosol foam or spray.

When the cosmetic composition according to the invention is used to protect hair, it can be provided in the form of a shampoo, lotion, gel, emulsion, non-ionic vesicular dispersion, and can constitute, for example, a rinse-out composition to be applied before of after shampooing, before or after dyeing or bleaching, before, during or after perming or hair-straightening, a styling or treating lotion or gel, a lotion or a gel for brushing or hair-setting, a composition for perming or hair-straightening, dyeing or bleaching the hair.

When the cosmetic composition according to the invention is used as a product for makeup of eyelashes, eyebrows or skin, such as cream for treating the epidermis, foundation, lipstick, eyelid makeup, cheek makeup, mascara or “liner” also known as “eye-liner”, it can be provided in an anhydrous or aqueous solid or paste form, such as emulsions of the oil-in-water or water-in-oil type, non-ionic vesicular dispersions or dispersions.

In order to further illustrate the present invention and the advantages thereof, the following specific examples are given, it being understood that same are intended only as illustrative and in nowise limitative.

In said examples to follow, all parts and percentages are given by weight, unless otherwise indicated.

### EXAMPLES

### Example 1

A material is prepared by the sol-gel route from the following ingredients:

| | |
|---|---|
| tetraethoxysilane (TEOS) | 5.0 g |
| water (HCl 0.1 N) | 0.87 g |
| Triton ® X-100 | 0.75 g |
| Parsol ® 1789 | 0.25 g |

with the tetraethoxysilane originating from Fluka, the Parsol® 1789 being marketed by HOFFMANN-LAROCHE, and the Triton® X-100 being marketed by Union Carbide.

The Parsol® 1789 is dispersed in the Triton® X-100 by heating to 40° C. for 30 minutes. Then the TEOS is added to this dispersion with stirring at ambient temperature. Next, acidified water is added dropwise. The whole is stirred for 24 hours at ambient temperature. The result is a material ready for use.

### COMPARATIVE EXAMPLE

A material is prepared by the sol-gel route from the following ingredients:

| | |
|---|---|
| tetraethoxysilane (TEOS) | 5.0 g |
| ethanol | 17 ml |
| water (HCl 0.1 N) | 0.9 g |
| Triton ® X-100 | 0.75 g |
| Parsol ® 1789 | 0.25 g |

with the tetraethoxysilane originating from Fluka, the Parsol® 1789 being marketed by HOFFMANN-LAROCHE, and the Triton® X-100 being marketed by Union Carbide.

The Parsol® 1789 is dispersed in the Triton® X-100 by heating to 40° C. for 30 minutes. Then the TEOS/ethanol is added to this dispersion with stirring at ambient temperature. Next, acidified water is added dropwise. The whole is stirred for 24 hours at ambient temperature. The result is a material ready for use.

Stability Tests with Respect to UV Radiation:

A test was then carried out on the stability with respect to UV radiation of Parsol® 1789 by irradiating the material obtained in Example 1 hereinabove as well as, by way of comparison, the material obtained in the comparative example.

Six glass plates (microscope plates) were prepared by depositing on each 100 μl of the material of Example 1. Films are formed after evaporation of the volatile components of the mixture. The drying time of the films is fast, that is to say, between 1 and 3 minutes. The material is then left as such at ambient temperature for 24 hours, before irradiation.

Three of the films obtained were irradiated by using an UVIMETRE OSTRAM-Centra equipped with a xenon lamp, under the following conditions:

power from the lamp UV-A=22 j/cm²,

irradiation times of the simulator=20 minutes.

These conditions correspond to approximately 1 hour of UV-A sunshine.

The photodecomposition of the Parsol® 1789 was evaluated by measuring the quantity of Parsol® 1789 before and after irradiation: the Parsol® 1789 was extracted from the films of each of the three non-irradiated plates and the three plaques irradiated with a set volume of ethanol by leaving the films for 24 hours and by stirring from time to time. In this way, all the Parsol® 1789 present in the films is extracted before and after irradiation, then the quantity of Parsol® 1789 is measured.

The results obtained for the material of Example 1 are presented in Table 1 below:

TABLE 1

| | mₚₐᵣₛₒₗ₁₇₈₉ (×10⁻³) g | | | | | | |
|---|---|---|---|---|---|---|---|
| | Plate No. | | | | | | % of Parsol ® 1789 |
| Material of Example 1 | 1 | 2 | 3 | 4 | 5 | 6 | decomposition |
| Before Irradiation | 3.1 | 3.4 | 3.1 | | | | |
| After Irradiation | | | | 3.1 | 2.8 | 3 | 7% |

The photodecomposition of the Parsol® 1789 in the material of the comparative example was evaluated by proceeding as for the material of Example 1. The results obtained are presented in Table 2 below:

TABLE 2

| | mₚₐᵣₛₒₗ₁₇₈₉ (×10⁻⁴) g | | | | | | |
|---|---|---|---|---|---|---|---|
| | Plate No. | | | | | | % of Parsol ® 1789 |
| Material of Example 1 | 1 | 2 | 3 | 4 | 5 | 6 | decomposition |
| Before Irradiation | 6.1 | 8 | 7.1 | | | | |
| After Irradiation | | | | 4.8 | .5 | 4.6 | 34% |

According to the results hereinabove, a clearly less significant photodecomposition (of the order of 7%) is obtained for the material according to the invention in comparison with that obtained with the material of the comparative Example 1 (on the order of 34%).

As for the material of Example 1, it has also been observed:

that the quality of the film is not altered by exposure to the sun;

low salting-out of the Parsol® 1789 in water and in a cosmetic oil, such as apricot oil and Parleam oil; and

good persistence of the film toward water.

Each patent, patent application, publication and literature article/report cited or indicated herein is hereby expressly incorporated by reference.

While the invention has been described in terms of various specific and preferred embodiments, the skilled artisan will appreciate that various modifications, substitutions, omissions, and changes may be made without departing from the spirit thereof. Accordingly, it is intended that the scope of the present invention be limited solely by the scope of the following claims, including equivalents thereof.

What is claimed is:

1. A photostable UV-photoprotecting amount of at least one dibenzoylmethane organic sunscreen immobilized within a matrix prepared via sol-gel process from at least one silicon alkoxide, at least one non-ionic surfactant and water, but in the absence of any organic solvent.

2. The matrix-immobilized dibenzoylmethane sunscreen as defined by claim 1, said at least one dibenzoylmethane sunscreen being selected from the group consisting of:
2-methyldibenzoylmethane,
4-methyldibenzoylmethane,
4-isopropyldibenzoylmethane,
4-tert-butyldibenzoylmethane,
2,4-dimethyldibenzoylmethane,
2, 5-dimethyldibenzoylmethane,
4,4′-diisopropyldibenzoylmethane,
4,4′-dimethyldibenzoylmethane,
4-tert-butyl-4′-methoxydibenzoylmethane,
2-methyl-5-isopropyl-4′-methoxydibenzoylmethane,
2-methyl-5-tert-butyl-4′-methoxydibenzoylmethane,
2,4-dimethyl-4′-methoxydibenzoylmethane, and
2,6-dimethyl-4-tert-butyl-4′-methoxydibenzoyl-methane.

3. The matrix-immobilized dibenzoylmethane sunscreen as defined by claim 2, said at least one dibenzoylmethane sunscreen comprising 4-tert-butyl-4′-methoxydibenzoylmethane.

4. The matrix-immobilized dibenzoylmethane sunscreen as defined by claim 1, said at least one silicon alkoxide having one of the following formulae (I), (II), (III) or (IV):
Si(ORₐ)₄ (I)
R—Si(ORₐ)₃ (II)
(RaO)₃—Si—R″—Si(ORₐ)₃ (III) 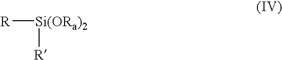
in which Rₐ is a linear or branched, C₁-C₃₀ alkyl radical; R and R′, which may be identical or different, are each a linear or branched alkyl radical, a cycloalkyl radical, a substituted or unsubstituted aryl radical, or said radicals R and R′ may be a cosmetically or dermatologically active substituent; and R″ is a linear or branched alkylene radical, a cycloalkylene radical, a substituted or unsubstituted arylene radical, or R″ may be a cosmetically or dermatologically active substituent.

5. The matrix-immobilized dibenzoylmethane sunscreen as defined by claim 4, said at least one silicon alkoxide comprising tetra-ethoxysilane or methyltriethoxysilane.

6. The matrix-immobilized dibenzoylmethane sunscreen as defined by claim 1, said at least one non-ionic surfactant being selected from the group consisting of polyethoxylated, polypropoxylated or polyglycerolated fatty alcohols, alkylphenols or acids comprising a fatty chain having from 6 to 32 carbon atoms, the number of ethylene oxide or propylene oxide structural units ranging from 2 to 100, and the number of glycerol structural units ranging from 2 to 30.

7. The matrix-immobilized dibenzoylmethane sunscreen as defined by claim 6, said at least one non-ionic surfactant comprising a polyethoxylated 4-(1,1,3,3-tetramethylbutyl)phenol.

8. A process for immobilizing and photostabilizing at least one dibenzoylmethane organic sunscreen within a sol-gel matrix, comprising admixing same with at least one non-ionic surfactant, at least one silicon alkoxide and water, in an amount and for such period of time sufficient for the partial or total hydrolysis of said at least one silicon alkoxide and condensation thereof, but in the absence of any organic solvent therefor.

9. The process as defined by claim 8, the amount of said at least one dibenzoylmethane organic sunscreen ranging from 0.1% to 20% by weight, relative to the weight of said at least one silicon alkoxide.

10. The process as defined by claim 8, the amount of said at least one dibenzoylmethane organic sunscreen ranging from 0.5% to 10% by weight, relative to the weight of said at least one silicon alkoxide.

11. The process as defined by claim 8, the weight ratio of said at least one surfactant to said at least one silicon alkoxide ranging from 0.01 to 99.

12. The process as defined by claim 8, the weight ratio of said at least one surfactant to said at least one silicon alkoxide ranging from 1 to 60.

13. The process as defined by claim 8, the water containing a cosmetically acceptable mineral or organic acid.

14. The process as defined by claim 8, the water of hydrolysis having a pH ranging from 1 to 4.

15. The process as defined by claim 8, the water of hydrolysis having a pH ranging from 1 to 2.

16. The process as defined by claim 13, comprising introducing said at least one silicon alkoxide into admixture of said at least one dibenzoylmethane sunscreen and said at least one non-ionic surfactant, and thence adding said acidified water thereto.

17. A photoprotective cosmetic/dermatological composition suited for the UV-photoprotection of the skin, hair, nails, eyebrows, eyelashes and/or lips, comprising an effective photostable and UV-photoprotecting amount of at least one dibenzoylmethane organic sunscreen immobilized within a matrix prepared via sol-gel process from at least one silicon alkoxide, at least one non-ionic surfactant and water, but in the absence of any organic solvent, formulated into a topically applicable, cosmetically/dermatologically acceptable medium therefor.

18 The cosmetic/dermatological composition as defined by claim 17, said at least one dibenzoylmethane sunscreen being selected from the group consisting of:
2-methyldibenzoylmethane,
4-methyldibenzoylmethane,
4-isopropyldibenzoylmethane,
4-tert-butyldibenzoylmethane,
2,4-dimethyldibenzoylmethane,
2,5-dimethyldibenzoylmethane,
4,4′-diisopropyldibenzoylmethane,
4,4′-dimethyldibenzoylmethane,
4-tert-butyl-4′-methoxydibenzoylmethane,
2-methyl-5-isopropyl-4′-methoxydibenzoylmethane,
2-methyl-5-tert-butyl-4′-methoxydibenzoylmethane,
2,4-dimethyl-4′-methoxydibenzoylmethane, and
2, 6-dimethyl-4-tert-butyl-4′-methoxydibenzoyl-methane.

19. The cosmetic/dermatological composition as defined by claim 18, said at least one dibenzoylmethane sunscreen comprising 4-tert-butyl-4′-methoxydibenzoylmethane.

20. The cosmetic/dermatological composition as defined by claim 17, said at least one silicon alkoxide having one of the following formulae (I), (II), (III) or (IV):
Si(ORₐ)₄ (I)
R—Si(ORₐ)₃ (II)
(RₐO)₃—Si—R″—Si(ORₐ)₃ (III) 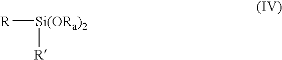
in which Rₐ is a linear or branched, C₁-C₃₀ alkyl radical; R and R′, which may be identical or different, are each a linear or branched alkyl radical, a cycloalkyl radical, a substituted or unsubstituted aryl radical, or said radicals R and R′ may be a cosmetically or dermatologically active substituent; and R″ is a linear or branched alkylene radical, a cycloalkylene radical, a substituted or unsubstituted arylene radical, or R″ may be a cosmetically or dermatologically active substituent.

21. The cosmetic/dermatological composition as defined by claim 20, wherein formulae (I), (II), (III) or (IV), Rₐ is a C₁-C₆ alkyl radical.

22. The cosmetic/dermatological composition as defined by claim 20, said at least one silicon alkoxide comprising tetra-ethoxysilane or methyltriethoxysilane.

23 The cosmetic/dermatological composition as defined by claim 17, said at least one non-ionic surfactant being selected from the group consisting of polyethoxylated, polypropoxylated or polyglycerolated fatty alcohols, alkylphenols or acids comprising a fatty chain having from 6 to 32 carbon atoms, the number of ethylene oxide or propylene oxide structural units ranging from 2 to 100, and the number of glycerol structural units ranging from 2 to 30.

24 The cosmetic/dermatological composition as defined by claim 23, said at least one non-ionic surfactant comprising a polyethoxylated 4-(1,1,3,3-tetramethylbutyl)phenol.

25. The cosmetic/dermatological composition as defined by claim 17, said matrix-immobilized dibenzoylmethane sunscreen comprising from 1% to 99% by weight, relative to the total weight of the composition.

26. The cosmetic/dermatological composition as defined by claim 17, said matrix-immobilized dibenzoylmethane sunscreen comprising from 1% to 60% by weight, relative to the total weight of the composition.

27. The cosmetic/dermatological composition as defined by claim 17, further comprising at least one additional organic sunscreen active in the UV-A and/or UV-B region, selected from the group consisting of anthranilates; salicylic derivatives; cinnamic derivatives; camphor derivatives; triazine derivatives; benzophenone derivatives; aminated 2-hydroxybenzophenones; β,β′-diphenylacrylate derivatives; benzotriazole derivatives; benzalmalonate derivatives; benzimidazole derivatives; imidazolines; bis(benzoazolyl) derivatives; p-aminobenzoic acid (PABA) derivatives; methylene-bis(hydroxyphenylbenzotriazole) derivatives; hydrocarbon polymer UV-filters and silicone UV-filters; alkylstyrene derivatives; and mixtures thereof.

28. The cosmetic/dermatological composition as defined by claim 27, said at least one additional organic sunscreen being selected from the group consisting of:
Ethylhexyl Salicylate,
Octocrylene,
Phenylbenzimidazole Sulfonic Acid,
Terephthalylidene Dicamphor Sulfonic Acid,
4-Methylbenzylidene Camphor,
Anisotriazine,
Ethylhexyl Triazone,
Diethylhexyl Butamido Triazone,
2,4,6-Tris-(diisobutyl-4′-aminobenzalmalonate)-s-triazine,
Benzophenone-3,
Benzophenone-4,
Benzophenone-5,
n-Hexyl 2-(4-diethylamino-2-hydroxybenzoyl)-benzoate,
Methylene bis-Benzotriazolyl Tetramethylbutyl-phenol,
Drometrizol Trisiloxane,
Disodium Phenyl Dibenzimidazole Tetrasulfonate, and mixtures thereof.

29. The cosmetic/dermatological composition as defined by claim 17, further comprising pigments or nanopigments of coated or uncoated metal oxides.

30. The cosmetic/dermatological composition as defined by claim 17, further comprising at least one cosmetic additive selected from the group consisting of fatty substances, organic solvents, ionic or non-ionic thickeners, softeners, antioxidants, agents for combating free radicals, opacifiers, stabilizers, emollients, silicones, α-hydroxy acids, anti-foaming agents, moisturizing agents, vitamins, insect repellents, perfumes, preservatives, anionic, cationic, amphoteric or non-ionic surfactants, anti-inflammatories, substance P antagonists, fillers, sequestering agents, polymers, propellants, basifying or acidifying agents, dyes, colorants, and mixtures thereof.

31. The cosmetic/dermatological composition as defined by claim 17, formulated as a non-ionic dispersion, emulsion, cream, gel, gel-cream, milk, stick, powder, pomade, foam or spray.

32. The cosmetic/dermatological composition as defined by claim 17, formulated as a shampoo or rinse.

33. The cosmetic/dermatological composition as defined by claim 17, formulated as a makeup.

34. A regime or regimen for photoprotecting the skin, hair, nails, eyebrows, eyelashes and/or lips against the damaging effects of UV radiation, comprising topically applying thereon a photoprotective cosmetic/dermatological composition comprising an effective photostable and UV-photoprotecting amount of at least one dibenzoylmethane organic sunscreen immobilized within a matrix prepared via sol-gel process from at least one silicon alkoxide, at least one non-ionic surfactant and water, but in the absence of any organic solvent, formulated into a topically applicable, cosmetically/dermatologically acceptable medium therefor.
